(19) United States
(12) Patent Application Publication
(10) Pub. No.: US 2021/0054343 A1
(43) Pub. Date: Feb. 25, 2021

(54) ENDOCARDIUM-DERIVED ADULT STEM CELLS AND METHOD FOR PRODUCING SAME

(71) Applicant: SEOUL NATIONAL UNIVERSITY HOSPITAL, Seoul (KR)

(72) Inventors: Hyo-Soo KIM, Seoul (KR); Han-Mo YANG, Seoul (KR); Ju-Young KIM, Suwon-si (KR); Hyun-Jai CHO, Seoul (KR)

(21) Appl. No.: 17/078,939

(22) Filed: Oct. 23, 2020

Related U.S. Application Data
(62) Division of application No. 14/888,415, filed on Oct. 30, 2015.

(30) Foreign Application Priority Data
Apr. 30, 2013 (KR) 10-2013-0048486

(51) Int. Cl. C12N 5/071; A61K 35/34; A61K 35/12; C12N 5/0775

(52) U.S. Cl. CPC C12N 5/0692; C12N 5/0668; A61K 35/12; A61K 35/34

(57) ABSTRACT

The present disclosure relates to endocardium-derived adult stem cells obtained by culturing peripheral blood mononuclear cells (PBMCs) separated from peripheral blood, and a cell therapeutic agent for treating cardiovascular diseases containing the same as an active ingredient. The adult stem cells have an origin that is the endocardium and strong blood vessel formation, and is thus remarkably useful for treating cardiovascular diseases such as ischemia, myocardial infarction, and the like.

### STATEMENT REGARDING GOVERNMENT RIGHTS

This invention was made with government support of Republic of Korea under Advanced Research Project (A062260) awarded by Korean Ministry of Health & Welfare. The government has certain rights in the invention. This invention was supported by the grants from the Bio and Medical Technology Development Program (2012M3A9C7050140) through the National Research Foundation of Korea (NRF).

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Technical Field

The present disclosure relates to an endocardium-derived adult stem cell obtained by culturing peripheral blood mononuclear cells (PBMCs) separated from peripheral blood and a cell therapeutic agent for treating vascular diseases containing the endocardium-derived adult stem cell as an active ingredient.

### Discussion of the Related Technology

Cells that can be used as a patient-specific cell therapeutic agent are classified into a somatic cell therapeutic agent and a stem cell therapeutic agent according to a cell type and a degree of differentiation. Among the agents, the stem cell therapeutic agent includes an adult stem cell therapeutic agent, an embryonic stem cell therapeutic agent, and a dedifferentiated stem cell therapeutic agent that has recently entered the spotlight as a replacement for the embryonic stem cells.

Adult stem cells are defined as cells that are found in developed body parts and organs of newborn babies or adults, have a self-renewal capability, and can be differentiated into various cells of biological tissues. When tissues or cells are damaged due to injuries or accidents, adult stem cells are differentiated into cells of muscles, bones, fat, nerves or the like, and can restore the damaged parts.

Adult stem cells include hematopoietic stem cells, mesenchymal stem cells, and tissue-specific progenitor cells having a limited differentiation ability involved in other tissue regeneration. Among them, cells that can be obtained using the most non-invasive method are hematopoietic stem cells or mesenchymal stem cells through bone marrow extraction, and the other cells are separated through a process of obtaining tissues in an invasive manner using a biopsy. Bone marrow extraction, though it is the most non-invasive method, requires anesthesia and causes pain. Therefore, as a further non-invasive method of separating patient-specific stem cells, a method obtaining cell using peripheral blood is required. However, when only peripheral blood is used, the number of hematopoietic stem cells or mesenchymal stem cells that can be separated from adults is limited, and the separating method costs. Even when cells are separated, the cells do not continuously proliferate to the extent available for cell treatment in many cases. Therefore, there is a need for alternative adult stem cells or a method obtaining cell through which practicability can be further increased.

In the related art, among tissue-specific progenitor cells that can be obtained through a biopsy, adult stem cells related to heart regeneration were identified in the pericardium and the myocardium. Stem cells obtained in the myocardium have a capability of forming the myocardium and coronary arteries, and have markers such as c-kit, Sca-1, side population, and Islet-1. Stem cells obtained in the pericardium have characteristics similar to those of mesenchymal stem cells, and can be differentiated into the myocardium or vascular smooth muscle cells. However, no adult stem cell was identified in the endocardium up to now, and the formation of cells the same as vascular endothelial cells was only considered. Such endocardial cells have functions of forming cardiac valves and blood vessels in the heart in development of the heart and have an NFATc1 marker, and expression of the NFATc1 marker gradually decreases with growth.

The disclosure of this section is to provide background information relating to the invention. Applicant does not admit that any information contained in this section constitutes prior art.

### SUMMARY

The inventors separated endocardium-derived multipotent adult stem cells from human peripheral blood, attempted to satisfy two needs, the provision of alternative adult stem cells and a non-invasive cell-obtaining method.

Specifically, an aspect of the present invention provides endocardium-derived adult stem cells to be used for patient-specific cell treatment through the collection of a small amount of peripheral blood using a non-invasive method, a cell therapeutic agent using the same and a method of preparing the same.

However, the scope of the present invention is not limited to the above-described aspects, and other aspects may be clearly understood by those skilled in the art from the following descriptions

Another aspect of the present invention provides endocardium-derived adult stem cells obtained when peripheral blood mononuclear cells (PBMCs) separated from peripheral blood are suspended in an EGM-2MV (Microvascular Endothelial Cell Growth Media-2) medium and seeded, and then T cells are removed and a culture is performed while the medium is exchanged daily for 5 to 8 days.

Still another aspect of the present invention provides a method of preparing endocardium-derived adult stem cells, including a step in which peripheral blood mononuclear cells (PBMCs) are separated from peripheral blood, and suspended in an EGM-2MV medium and seeded; and a step in which T cells are removed and a culture is performed while the medium is exchanged daily for 5 to 8 days after the seeding,

According to a specific example of the present invention, the adult stem cell has the following characteristics: (a) a positive immunological characteristic for NFATc1, MixL1 and CD31 and a negative immunological characteristic for WNT5A and CD3; and (b) growing in an adherent manner and showing a morphologic characteristic of a spindle shape

One embodiment of the present invention provides a cell therapeutic agent for treating vascular diseases, containing the adult stem cell as an active ingredient.

Another embodiment of the present invention provides a method of preventing or treating vascular diseases, including administering the adult stem cell of a pharmaceutically effective amount to a subject.

Still another embodiment of the present invention provides a method of using the adult stem cell to prevent or treat vascular diseases.

As a specific example of the present invention, the vascular diseases include myocardial infarction or lower limb ischemia.

Stem cells according to embodiments of the present invention are originated from the endocardium rather than the pericardium and myocardium studied in the related art, have multipotency, and can be separated from peripheral blood of only a small amount and cultured. Since cells and an environment suppressing adult stem cells are removed only when the medium is simply and repeatedly exchanged in a culture process, it can easily prepared.

Also, since stem cells according to embodiments of the present invention have a high proliferative ability, it is possible to ensure cells (1×10⁷ cells) that are stored without genetic variation within one month after the culture and can be used for cell treatment.

Also, since stem cells according to embodiments of the present invention can induce blood vessel formation due to a proliferative ability and differentiation ability of cells themselves when the cells are injected into tissues, the cells can be used for treating diseases in which ischemia is induced, have higher gene introduction efficiency than immune cells, and can be differentiated into other types of cells after gene introduction and used for treatment.

Also, since stem cells according to embodiments of the present invention are originated from the endocardium and have a high vasculogenic ability, the stem cells can greatly contribute to mechanism research and treatment of cardiovascular diseases such as myocardial infarction.

### BRIEF DESCRIPTION OF THE DRAWINGS

FIG. 1a-1c show a culture method of CiMS stem cells (FIG. 1a), cell types according to an appearance time of CiMS stem cells (FIG. 1b), and a surface marker of CiMS stem cells (FIG. 1c).

FIG. 2a shows an inhibitory effect of a CiMS stem cell culture due to T lymphocytes through colony staining and FIG. 2b shows a percentage of CD3+ cells in suspended cells in an initial CiMS culture.

FIG. 3a shows the analyzed result of CiMS genotypes of patients who had undergone bone marrow transplantation, liver transplantation, or kidney transplantation, and FIG. 3b shows the analyzed result of CiMS genotypes obtained from blood of heart donors and recipients.

FIG. 4a shows positions of NFATc1 in CiMS using immunostaining and FIG. 4b shows the RT-PCR result in which a CiMS-specific marker is identified.

FIG. 5 shows the staining result of NFATc1 and CD31 markers in heart tissues.

FIG. 6 shows the result of a CiMS culture using staining of NFATc1/CD31, which are CiMS-specific markers in peripheral blood mononuclear cells (PBMCs).

FIG. 7a shows the comparison result of appearance times of CiMS between healthy volunteers and heart transplant patients, and FIG. 7b shows the result of the number of cells expressing a CiMS marker, (NFATc1+/CD31+/CD3−), in healthy volunteers and heart transplant patients.

FIG. 8a shows an effect of CiMS injection in a mouse myocardial infarction model, FIG. 8b shows a distribution of GFP-CiMS in myocardial infarction heart tissues, and FIG. 8c shows an effect of CiMS injection in a mouse lower limb ischemia model.

### DETAILED DESCRIPTION OF EMBODIMENTS

Embodiments of the present invention relate to an endocardium-derived multipotent stem cell separated from peripheral blood mononuclear cells (PBMCs) in peripheral blood.

In embodiments of the present invention, the term “stem cells” refer to cells that form a subject or are the foundation of tissues, and have characteristics such as repeatedly dividing and self-renewal, and multipotency of differentiating into cells having a specific function according to an environment. Stem cells are generated in all tissues during fetal developmental processes, and found in some tissues in which cells are actively replaced such as bone marrow and epithelial tissues in adults. Stem cells are divided into totipotent stem cells that are formed when a first division of an embryo starts, pluripotent stem cells in an inner membrane of the blastocyst that is formed by repeated divisions of the cells and multipotent stem cells included in mature tissues and organs according to types of cells that can be differentiated. In this case, the multipotent stem cells are cells that can be differentiated only into cells specific to tissues and organs including the cells, are involved in growth and development of tissues and body parts of fetal stages, neonatal stages and adult stages, homeostatic maintenance of adult tissues and functions of inducing regeneration when tissues are damaged. Such tissue-specific multipotent cells are collectively referred to as “adult stem cells.”

In embodiments of the present invention, the term “peripheral blood” refers to blood that circulates in bodies of mammals (including humans), and can be diversely extracted using arteries, veins, peripheral blood vessels or the like.

In embodiments of the present invention, the term “peripheral blood mononuclear cell (PBMC)” refers to a mononuclear cell present in peripheral blood, and includes immune cells such as B cells, T cells, macrophages, dendritic cells, and natural killer (NK) cells, and granulocytes such as a basophil, eosinophil, and neutrophil. The PBMC can be separated using general preparing methods, for example, density gradient centrifugation using, for example, Ficoll-Paque (Blood, 1998, 92: 2989-93, etc.).

In embodiments of the present invention, a method of preparing endocardium-derived stem cells, preferably, PBMCs are separated from peripheral blood, are suspended and seeded in an EGM-2MV medium (Microvascular Endothelial Cell Growth Media-2) (Lonza; Basel, Switzerland), and the medium is exchanged daily for the first 5 days such that no colony including T cells is formed. In this manner, when the medium is exchanged daily, several cells are observed between about the 5th day to the 8th day, and proliferated to an amount of cells that can be sub-cultured and maintained within 2 weeks. These cells are called “circulating multipotent stem cells (CiMSs).” In CiMSs, markers of mesenchymal stem cells such as SH2, SH3, CD13, CD29, CD44, and HLA-ABC and an endothelial cell surface marker of CD31 were expressed, and markers such as CD14, CD34, CD45, and HLA-DR were not expressed. The CiMS had a property different from a bone marrow-derived blood cell.

In embodiments of the present invention, the CiMS shows growing in an adherent manner and a morphologic characteristic of a spindle shape. In this case, the term “adherent” refers to adhering to a culture flask, a plastic or the like and growing and an adherence target is not limited.

In embodiments of the present invention, an endocardium-derived stem cell can be prepared using a simple culture method in which T cells (lymphocytes) suppressing an appearance of specific adult stem cells in PBMCs are removed.

In embodiments of the present invention, in order to determine the origin of the CiMS, the blood of patients who had undergone bone marrow transplantation, patients who had undergone liver transplantation, patients who had undergone kidney transplantation and patients who had undergone heart transplantation were cultured, genotypes of donors and genotypes of recipients were identified using an STR test. As a result, all of the obtained CiMSs had genotypes identical to those of the recipients except the CiMS of the patients who had undergone heart transplantation, and only the CiMS cultured from the patients who had undergone heart transplantation had genes identical to the donor.

In the CiMS herein, NFATc1, which is a myocardium endometrial cell marker, was significantly expressed compared to other endothelial cells, mesenchymal stem cells, skin fibroblasts, and blood cells, and MixL1, which is a marker of a primitive streak and a mesoderm, was expressed, but Wnt5a, which is a marker related to proliferation or migration of endothelial cells, was not expressed. Using such findings, heart tissues were obtained from patients who would undergo heart transplantation and tissue staining was performed. As a result, since cells simultaneously expressing NFATc1 and CD31 were in some places of the endocardium, the origin of the CiMS was determined as the endocardium.

Immunostaining was performed using the CiMS prepared in embodiments of the present invention. As a result, a cytoplasm was stained with NFATc1. Therefore, the inventors established a method in which PBMCs were separated from blood and streptolysin O (SLO) was used to set a condition in which anti-NFATc1 antibodies can permeate a cell membrane, an NFATc1/CD31 double positive group was cultured in a CD3 negative group, and thus the CiMS can be directly selected and cultured. The result identified through such a method showed that, heart transplant patients whose CD3 cell functions further significantly decreased due to administration of immunosuppressive agents had a greater amount of the CiMS separated from blood than normal persons and an appearance time thereof in a culture decreased.

The CiMS according to embodiments of the present invention has multipotency. The CiMS stained with GFP was injected into the heart of a myocardial infarction-induced mouse and a lower limb muscle of an ischemia-induced mouse. As a result, from the 14th day onward, it can be observed that the CiMS-injected group had further improved left ventricle contractility and blood circulation than a control group and it can be observed that green fluorescent cells were differentiated into endothelial cells and vascular smooth muscle cells in the heart of the mouse and formed a blood vessel. Therefore, it can be directly seen that a main function of the CiMS is restoration of damaged blood vessels.

Hereinafter, embodiments of the present invention will be described in further detail through examples. However, the following examples are only examples of the present invention, and the present invention is not limited to the following examples.

### EXAMPLES

### Example 1. Establishment of Separating and Culturing Stem Cell from Peripheral Blood

### Example 1-1. CiMS Culture Method Using Peripheral Blood

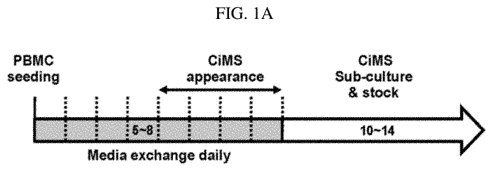

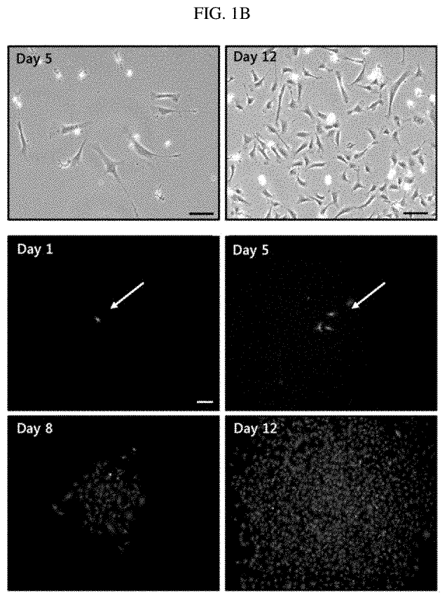

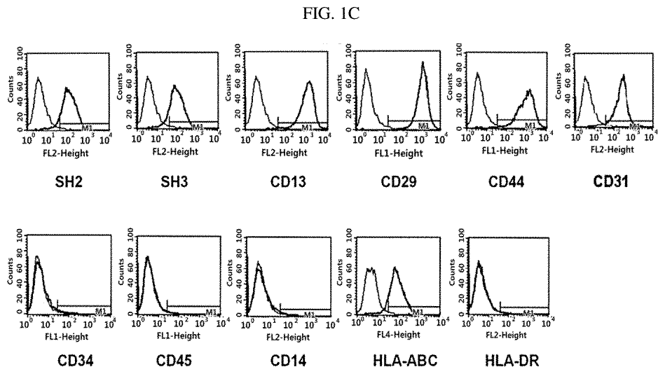

PBMCs were separated from human blood using Ficoll-Paque, suspended in an EGM-2MV medium (Lonza; Basel, Switzerland), and seeded in a 6-well plate coated with 10 μg/ml fibronectin such that each well had 4×10⁶ PBMCs/ml, and then cultured in a 5% CO₂ incubator. The plate was shaken several times and floating cells were removed through strong suctioning the next day. Then, a procedure in which the medium was exchanged with a new medium and a culture was performed was repeated for 7 days. From the 7th day onward, the medium was exchanged once every two days. As a result, as shown in FIGS. 1a and 1b, an appearance of the CiMS cell was identified between the 5th day to the 8th day, and a colony was formed and proliferated within 2 weeks after the appearance of the CiMS cell. This colony was sub-cultured using 0.05% trypsin/EDTA, suspended in an FBS stock medium including 10% DMSO, input to an isopropanol freezing container, left for 24 hours at −70° C., and then maintained at −190° C.

### Example 1-2. Identification of Marker of Separated CiMS

In order to identify a surface marker of the CiMS cell obtained in Example 1-1, flow cytometry was performed. As a result, as shown in FIG. 1c, in the CiMS, markers of mesenchymal stem cells such as SH2, SH3, CD13, CD29, CD44, and HLA-ABC, and an endothelial cell surface marker of CD31 were expressed, and markers of bone marrow-derived blood cells such as CD14, CD34, CD45, and HLA-DR were not expressed.

### Example 2. Identification of Suppressing Effect of Stem Cell Culture Due to T Lymphocytes

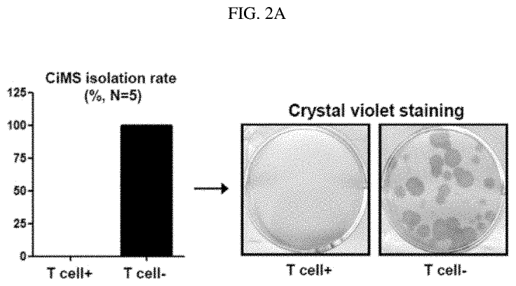

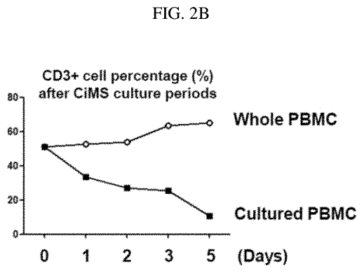

In PBMCs, a pan T MACS (Magnetic-activated cell sorting) separation kit was used to divide samples into a group in which T lymphocytes were removed and a group in which T lymphocytes were included, and then a CiMS culture was performed while suspended cells were present without repeatedly changing the medium. Then, a CiMS colony was stained with crystal violet, and a CiMS appearance according to addition of T lymphocytes was identified. As a result, as shown in FIG. 2a, a phenomenon in which an appearance of the CiMS is inhibited was observed in the culture group in which T lymphocytes are included, which shows that T lymphocytes suppressing an appearance of the CiMS were removed when the medium is repeatedly changed.

Also, suspended cells of a supernatant were obtained daily, T lymphocytes were stained with CD3 antibodies, and then flow cytometry was performed. Accordingly, a percentage of T lymphocytes was identified in suspended cells that were initially removed in the CiMS culture. FIG. 2b shows the result.

### Example 3. Identification of Origin of CiMS

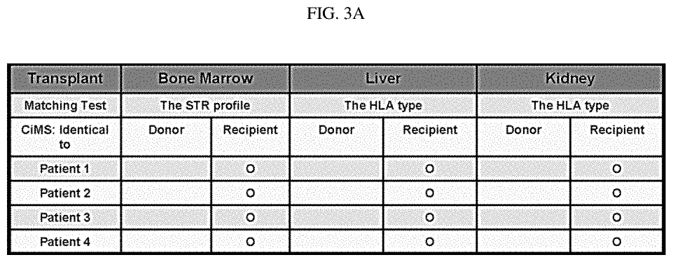

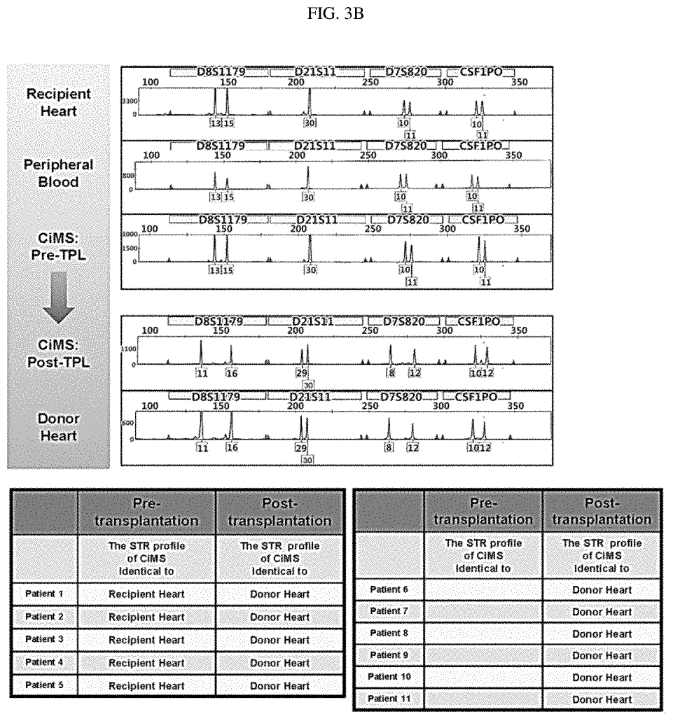

In order to determine the origin of the CiMS, CiMSs were obtained from PBMCs of patients who had undergone bone marrow transplantation, patients who had undergone liver transplantation, and patients who had undergone kidney transplantation using the method of Example 1-1, and then it was identified whether a genotype is identical to that of a recipient or a donor using the STR test and HLA typing. As a result, as shown in FIG. 3a, since the CiMS had a genotype identical to that of the recipient, it can be seen that the CiMS is not originated from the bone marrow, the liver, or the kidney.

Also, a genotype of the CiMS obtained from PBMCs of the patients who had undergone heart transplantation was analyzed using the STR test. As a result, as shown in FIG. 3b, it was observed that the CiMS has a genotype identical to that of the donor. When pre-heart transplantation and post-heart transplantation were compared, a phenomenon in which the CiMS having a genotype identical to that of the recipient before heart transplantation was changed to the CiMS having a genotype identical to that of the donor after heart transplantation was observed in 11 patients. This proves the fact that the origin of the CiMS is the heart.

### Example 4. Analysis of Endocardium-Specific Gene Expression of CiMS

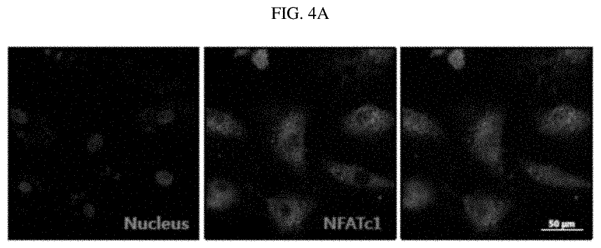

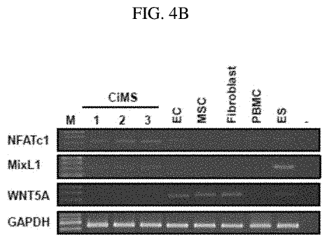

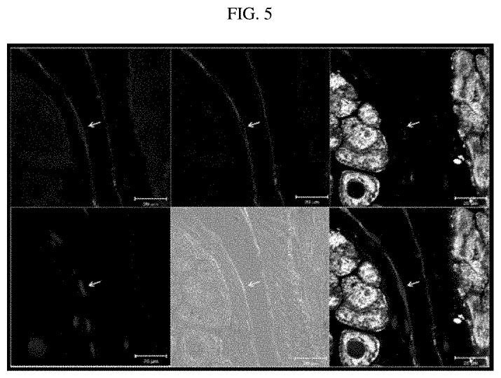

In order to determine a position of the CiMS in the heart, it may be necessary to develop a CiMS-specific marker. For this purpose, immunostaining was performed on the CiMS. As a result, as shown in FIG. 4a, it can be seen that NFATc1 is in the cytoplasm of the CiMS. That is, it can be observed that, through screening of several genes, in the CiMS, NFATc1, which is a myocardium endometrial cell marker that is specifically expressed in myocardium endometrial cells rather than other endothelial cells, mesenchymal stem cells, skin fibroblasts, blood cells, and embryonic stem cells, was significantly expressed. Therefore, NFATc1 was determined as a cell-specific marker of the CiMS.

Also, as shown in FIG. 4b, it can be seen that, in the CiMS, MixL1, which is a marker of a primitive streak and a mesoderm, was expressed, and thus the CiMS has a property of progenitor cells, and WNT5A, which is a marker related to proliferation or migration of endothelial cells, was not expressed, and thus WNT5A can be used as a negative marker.

### Example 5. Verification of the Presence of CiMS Through NFATc1 and CD31 Staining in Endocardium

Heart tissues were obtained from the patients who had undergone heart transplantation, and then immunohistologic staining using a CiMS-specific marker was performed by the following method. The heart tissues were fixed with paraformaldehyde (PFA) to make paraffin blocks, subjected to a deparaffinization process, immersed in a DAKO retrieval solution, subjected to a retrieval process in a microwave, and then staining was performed. Antibodies for NFATcz1 were conjugated with digoxigenin and used (1:100) using a Solulink Chromalink digoxigenin one-shot antibody labeling kit for signal amplification and specificity. Antibodies for CD31 were conjugated with biotin and used (1:100). As shown in FIG. 5, in the immunostaining result, it can be seen that cells simultaneously expressing NFATc1 and CD31 are in the endocardium rather than the pericardium or the myocardium (refer to the arrows). Therefore, it can be seen that the position of the CiMS in the heart is the endocardium.

### Example 6. Culture of CiMS Using CiMS-Specific Marker (NFATc1/CD31)

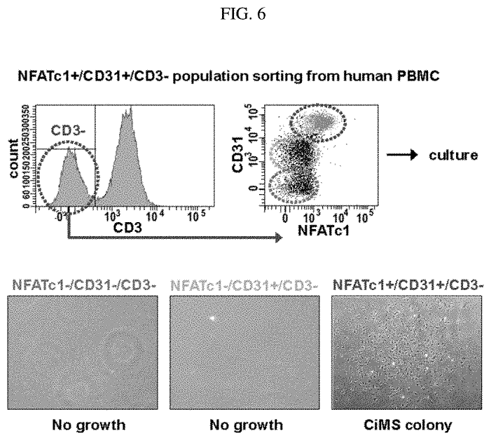

Based on the result that NFATc1 is in the cytoplasm of the CiMS, a method in which the CiMS is directly separated from PBMCs using the NFATc1 marker was developed. First, PBMCs were separated from blood and treated with SLO to set a condition in which NFATc1 antibodies can permeate a cell membrane. Then, CD31 and CD3 antibodies were adhered, a cell group which was significantly stained with NFATc1 and CD31 at the same time was separated from the CD3 negative groups through sorting using flow cytometry and cultured. As a result, as shown in FIG. 6, in culture groups of NFATc1−/CD31−/CD3−, NFATc1−/CD31+/CD3−, and NFATc1+/CD31+/CD3−, a CiMS colony was identified only in the NFATc1+/CD31+/CD3− group. Therefore, the method in which the CiMS can be directly selected from PBMCs and cultured was established.

### Example 7. Analysis of Appearance Time and Amount of CiMS in Peripheral Blood

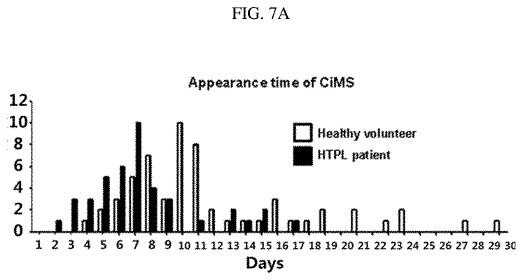

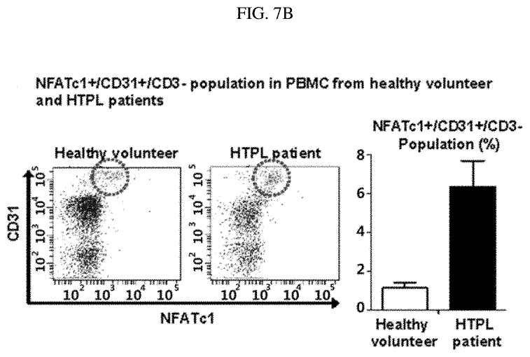

CiMSs were obtained from healthy volunteers (control group) and heart transplant patients (HTPL patients) and cultured. Appearance times thereof were compared and analyzed. As a result, as shown in FIG. 7a, it can be seen that the CiMS appeared in heart transplant patients (42 persons) whose T lymphocyte functions significantly decreased due to administration of immunosuppressive agents an average of 2 days earlier than in healthy volunteers (58 persons).

Also, CiMSs were obtained from healthy volunteers and heart transplant patients and cultured, and flow cytometry was performed using a CiMS-specific marker (NFATc1+/CD31+/CD3−). As a result, as shown in FIG. 7b, it can be seen that the number of CiMSs of the heart transplant patients increased about four times more than in the healthy volunteer.

### Example 8. Determination of Roles of CiMS in Mouse Myocardial Infarction Model and Lower Limb Ischemia Model

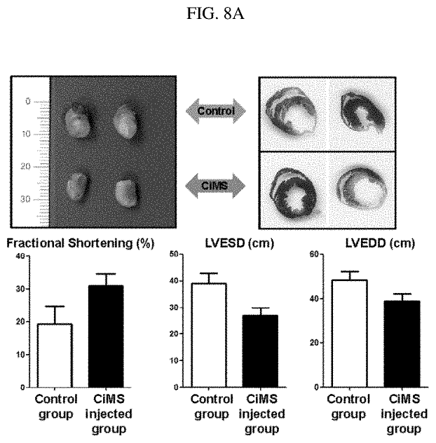

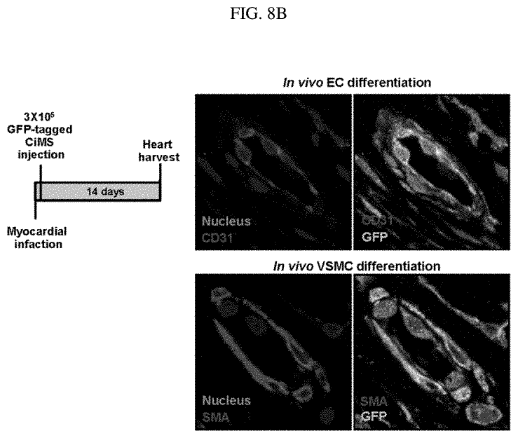

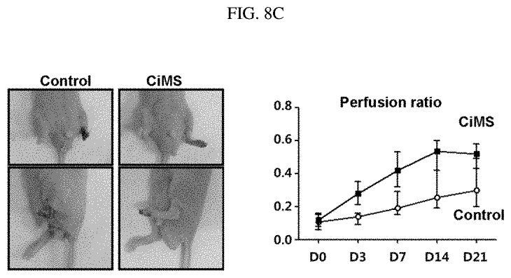

In order to determine roles of the CiMS in the mouse myocardial infarction model, CiMSs (3×10⁶ cells) labeled with green fluorescent proteins (GFPs) were injected into the heart of a myocardial infarction-induced mouse. As a result, as shown in FIG. 8a, from the 14th day onward, in a control group in which no CiMS was injected, a size of the myocardial infarction was 19.52%, and in a group in which CiMSs were injected, a size of the myocardial infarction was 8.23%. It was observed that injection of CiMSs has an effect of significantly decreasing the myocardial infarction. Also, it can be observed that left ventricle contractility obtained by measuring a left ventricle inner diameter fraction rate (fractional shortening), a left ventricular end-diastolic diameter (LVESD), and a left ventricular end-systolic diameter (LVEDD) increased in the CiMS-injected group more than in the control group. Also, the heart tissue of the myocardial infarction-induced mouse was analyzed. As a result, as shown in FIG. 8b, it can be seen that CiMSs were generally differentiated into endothelial cells (ECs) and vascular smooth muscle cells (VSMCs), and formed a blood vessel.

Also, in order to determine roles of the CiMS in the mouse lower limb ischemia model, CiMSs (3×10⁶ cells) labeled with green fluorescent proteins (GFPs) were injected into lower limb muscles of a lower limb ischemia-induced mouse. As a result, as shown in FIG. 8c, from the 14th day onward, the group in which CiMSs were injected showed more excellent lower limb regeneration than the control group in which no CiMS was injected. When a perfusion ratio was measured using a laser Doppler instrument, it was observed that the CiMS-injected group had an increased perfusion ratio more than the control group.

Based on these results, it can be seen that a main function of the CiMS in tissues is restoration of damaged blood vessels and regeneration of tissues.

Stem cells according to embodiments of the present invention are originated from the endocardium rather than the pericardium and myocardium studied in the related art, have multipotency, and can be separated from peripheral blood of only a small amount and cultured. Since cells and an environment suppressing adult stem cells are removed only when the medium is simply and repeatedly exchanged in a culture process, it can easily prepared. Also, stem cells according to embodiments of the present invention can greatly contribute to mechanism research and treatment of cardiovascular diseases such as myocardial infarction.

What is claimed is:

1. A method of preparing the endocardium-derived adult stem cell, comprising:
(a) separating peripheral blood mononuclear cells (PBMCs) from peripheral blood, then suspending in an EGM-2MV (Microvascular Endothelial Cell Growth Media-2) medium, and then seeding; and
(b) subsequent to seeding, culturing while exchanging the medium for 5 to 8 days after the seeding,
wherein T cells are removed from the medium while culturing, and
wherein the endocardium-derived adult stem cell has the following characteristics,
(a) a positive immunological characteristic for NFATc1 and CD31 and a negative immunological characteristic for CD3;
(b) growing in an adherent manner and showing a morphologic characteristic of a spindle shape;
(c) having multipotency; and
(d) capable of forming blood vessels.

2. The method according to claim 1,
wherein furthermore, the endocardium-derived adult stem cell has the following characteristic,
a positive immunological characteristic for MixL1 and a negative immunological characteristic for WNT5A.

3. The method according to claim 1,
wherein the endocardium-derived adult stem cell is used as cell therapeutic agent for treating vascular diseases.

4. The method according to claim 3,
wherein the vascular diseases include myocardial infarction or lower limb ischemia.
